(19) United States
(12) Patent Application Publication
(10) Pub. No.: US 2004/0229839 A1
(43) Pub. Date: Nov. 18, 2004

(54) Substituted nucleosides, preparation thereof and use as inhibitors of RNA viral polymerases

(75) Inventors: Yarlagadda S. Babu, Birmingham, AL (US); Pooran Chand, Birmingham, AL (US); Yahya El-Kattan, Hoover, AL (US)

Correspondence Address: CONNOLLY BOVE LODGE & HUTZ LLP, SUITE 800, 1990 M STREET NW, WASHINGTON, DC, 20036-3425, US

(73) Assignee: BIOCRYST PHARMACEUTICALS, INC., Birmingham, AL (US)

(21) Appl. No.: 10/845,233

(22) Filed: May 14, 2004

Related U.S. Application Data
(60) Provisional application No. 60/470,193, filed on May 14, 2003.

(51) Int. Cl. C07H 19/04; A61K 31/675; A61K 31/7076; A61K 31/7072

(52) U.S. Cl. 514/47; 514/81; 514/50; 514/85; 536/26.1; 544/243; 544/244

(57) ABSTRACT

Provided are compounds represented by:

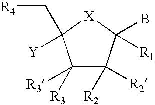

X is O, S or NR₆,

R₁ is H or (CH₂)ₘR₅,

R₂, R_{2′}, R₃ and R_{3′} are independently NO₂, N₃ or (CH₂)ₘR₅, OH

R₄ is H, OR₆, SR₆, NR₆R₆ₐ, CN, C(O)OR₆, C(O)NR₆R₆ₐ, R₆, OR₇ or (CH₂)ₙR₇,

R₅ is H, halo, OR₆, SR₆, NR₆R₆ₐ, CN, C(O)OR₆, C(O)NR₆R₆ₐ, R₆, OR₇ or (CH₂)ₘR₇,

R₆ and R₆ₐ are individually H, alkyl, substituted alkyl, alkenyl, substituted alkenyl, alkynyl, substituted alkynyl, aryl or substituted aryl,

R₇ is:

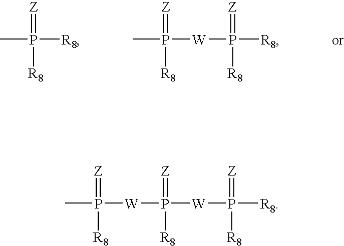

R₈ is H, F, SR₉ or OR₉,

R₉ is H, alkyl, alkenyl, alkynyl, aryl or hydroxyprotecting group,

Y is H, CH₃ or (CH₂)ₘR₅,

Z is O or S

W is CH₂, CF₂, CHF or O,

m is 0-4,

B is adenine, guanine, cytosine, uracil, thymine, modified purines and pyrimidines substituted pyridines, five membered heterocycles substituted by at least one of amines, substituted amines, amides, substituted amides, esters, halogens, alkyls, ethers; and pharmaceutically acceptable salts thereof and prodrugs thereof. These ring systems may be substituted.

### TECHNICAL FIELD

The present invention relates to certain nucleosides and particularly to nucleosides that are useful as inhibitors of viral RNA polymerases such as, but not limited to, hepatitis B, hepatitis C, Polio, Coxsackie A and B, Rhino, Echo, small pox, Ebola, and West Nile virus polymerases.

The present invention also relates to pharmaceutical compositions comprising the composition of the present invention, as well as methods of using the compounds in inhibiting viral RNA polymerases and treating patients suffering from diseases caused by various RNA viruses.

The present invention also relates to a method for producing the compounds of the present invention.

### BACKGROUND OF THE INVENTION

Hepatitis C virus (HCV), as a particular example of an RNA virus, has infected an estimated 170 million people worldwide, leading to a major health crisis as a result of the disease. Indeed, during the next few years the number of deaths from HCV-related liver disease and hepatocellular carcinoma may overtake those caused by AIDS. Egypt is the hardest hit country in the world, with 23% of the population estimated to be carrying the virus; whereas, in the USA the prevalence of chronic infections has recently been determined to be around 1.87% (2.7 million persons). HCV infections become chronic in about 50% of cases. Of these, about 20% develop liver cirrhosis that can lead to liver failure, including hepatocellular carcinoma.

The NS5B region of HCV encodes a 65 KDa RNA-dependent RNA polymerase (RdRp) thought to be responsible for viral genome replication. RdRps function as the catalytic subunit of the viral replicase required for the replication of all positive-strand viruses. The NS5B protein has been well characterized, shown to possess the conserved GDD motif of RNA-dependent RNA polymerases and in vitro assay systems have been reported. Cellular localization studies revealed that NS5B is membrane-associated in the endoplasmic reticulum like NS5A, suggesting that those two proteins may remain associated with one another after proteolytic processing. Additional evidence suggests that NS3, NS4A and NS5B interact with each other to form a complex that functions as part of the replication machinery of HCV.

The X-ray crystal structure of NS5B apoenzyme has now been determined and three very recent publications describe the unusual shape of the molecule. This unique shape for a polymerase, resembling a flat sphere, is attributed to extensive interactions between the fingers and thumb subdomains in such a way that the active site is completely encircled, forming a cavity 15 Å across and 20 Å deep. Modeling studies showed that the NS5B apoenzyme can accommodate the template-primer without large movement of the subdomains, suggesting that the structure is preserved during the polymerization reaction. Hopefully the information gained from future co-crystallization work with template-primer, NTP and inhibitors will lead to rapid progress in drug discovery for this target.

There are only a few reports of weak inhibitors of the polymerase. These include some nucleotide analogues, gliotoxin and the natural product cerulenin.

Bovine viral diarrhea virus (BVDV) has been used as a model for the identification of nucleoside analogues as candidate inhibitors of the polymerase. Active compounds from a BVDV cell-based assay were prepared as triphosphates and tested against the isolated NS5B polymerase enzyme. Unfortunately, none of these compounds directly inhibited the polymerase when tested in vitro as triphosphates.

Accordingly, it would be desirable to develop inhibitors of RNA viral polymerases.

### SUMMARY OF INVENTION

In particular, the present invention relates to compounds represented by the formula:

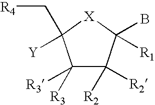

X is chosen from O, S, and NR₆,

R₁ is selected from the group consisting of H, and (CH₂)ₘR₅

R₂, R_{2′}, R₃ and R_{3′} are independently chosen from NO₂, N₃, and (CH₂)ₘR₅, OH

R₄ is selected from the group consisting of H, OR₆, SR₆, NR₆R₆ₐ, CN, C(O)OR₆, C(O)NR₆R₆ₐ, R₆, OR₇, and (CH₂)ₘR₇

R₅ is selected from the group consisting of H, halo, OR₆, SR₆, NR₆R₆ₐ, CN, C(O)OR₆, C(O)NR₆R₆ₐ, R₆, OR₇, and (CH₂)ₘR₇,

R₆ and R₆ₐ are individually selected from the group consisting of H, alkyl, substituted alkyl, alkenyl, substituted alkenyl, alkynyl, substituted alkynyl, aryl, and substituted aryl, R₇ is chosen from:

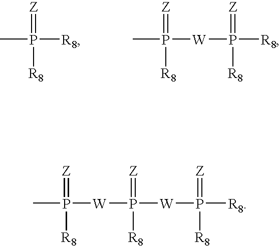

R₈ is selected from the group consisting of H, F, SR₉, and OR₉

R₉ is chosen from the group of H, alkyl, alkenyl, alkynyl, aryl, and hydroxyprotecting group,

Y is chosen from H, CH₃, and (CH₂)ₘR₅,

Z is chosen from O and S

W is chosen from CH₂, CF₂, CHF, and O,

m is 0, 1, 2, 3, or 4,

B is selected from adenine, guanine, cytosine, uracil, thymine, modified purines and pyrimidines such as inosin-9-yl, 2-amino-purin-9-yl, 2-amino-6-chloro-purin-9-yl, 2-6-diamino-purin-9-yl, 3-carboxamido-1,2,4-triazol-1-yl, 3-deaza-adenin-9-yl, 3-deaza-guanin-9-yl, 3-deaza-inosin-9-yl, 3-deaza-2-amino-purin-9-yl, 3-deaza-2-amino-6-chloro-purin-9-yl, 3-deaza-2,6-diamino-purin-9-yl, 7-deaza-adenin-9-yl, 7-deaza-guanin-9-yl, 7-deaza-inosin-9-yl, 7-deaza-2-amino-purin-9-yl, 7-deaza-2-amino-6-chloro-purin-9-yl, 7-deaza-2-6-diamino-purin-9-yl, 7-deaza-8-aza-adenin-9-yl, 7-deaza-8-aza-guanin-9-yl, 7-deaza-8-aza-inosin-9-yl, 7-deaza-8-aza-2-amino-purin-9-yl, 7-deaza-8-aza-2-amino-6-chloro-purin-9-yl, 7-deaza-8-aza-2-6-diamino-purin-9-yl, 8-aza-adenin-9-yl, 8-aza-guanin-9-yl, 8-aza-inosin-9-yl, 8-aza-2-amino-purin-9-yl, 8-aza-2-amino-6-chloro-purin-9-yl, 8-aza-2-6-diamino-purin-9-yl, 5-aza-thymin-1-yl, 5-aza-cytosin-1-yl, 5-aza-uracil-1-yl, 6-aza-thymin-1-yl, 6-aza-cytosin-1-yl, 6-aza-uracil-1-yl, 2-thiouracil-1-yl, 4-thiouracil-1-yl, 2-thiocytosine-1-yl, uracil-5-yl, 2-thiouracil-5-yl, 4-thiouracil-5-yl, substituted pyridine derivatives, 6-azauracil, azacytosine, five membered heterocycles with one to three heteroatoms (examples but not limited to triazole, thiazole, pyrazole, imidazole, pyrrole, thiophene, furane) substituted by at least one member selected from the group consisting of amines, substituted amines, amides, substituted amides, esters, halogens, alkyls, ethers; and pharmaceutically acceptable salts thereof and prodrugs thereof. In general, attachment may be at different positions in the ring at nitrogen or carbon. These ring systems may be substituted with halo, alkyl, substituted alkyl (F, Cl, Br, I, OH), NH₂, N₃, aryl, substituted aryl (F, Cl, Br, I, OH, NH₂), and aralkyl.

Another aspect of the present invention relates to pharmaceutical composition containing at least one of the above-disclosed compounds.

The present invention also relates to a method for inhibiting RNA polymerases in a patient by administering to the patient at least one of the above-disclosed compounds in an amount sufficient to inhibit viral RNA polymerases, such as HCV, small pox, Ebola virus, and West Nile virus.

The present invention is also concerned with methods of using the compounds of the present invention in treating a patient suffering from RNA viral infections such as HCV, HBV and Rhino viral infection by administering to the patient an effective amount of at least one of the above-disclosed compounds.

Still other objects and advantages of the present invention will become readily apparent by those skilled in the art from the following detailed description, wherein it is shown and described preferred embodiments of the invention, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### BEST AND VARIOUS MODES FOR CARRYING OUT INVENTION

In particular, the present invention relates to compounds represented by the formula:

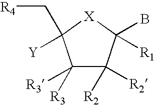

X is chosen from O, S, and NR₆,

R₁ is selected from the group consisting of H, and (CH₂)ₘR₅

R₂, R_{2′}, R₃ and R_{3′}, are independently chosen from NO₂, N₃, and (CH₂)ₘR₅, OH

R₄ is selected from the group consisting of H, OR₆, SR₆, NR₆R₆ₐ, CN, C(O)OR₆, C(O)NR₆R₆ₐ, R₆, OR₇, and (CH₂)ₘR₇

R₅ is selected from the group consisting of H, halo, OR₆, SR₆, NR₆R₆ₐ, CN, C(O)OR₆, C(O)NR₆R₆ₐ, R₆, OR₇, and (CH₂)ₘR₇,

R₆ and R₆ₐ are individually selected from the group consisting of H, alkyl, substituted alkyl, alkenyl, substituted alkenyl, alkynyl, substituted alkynyl, aryl, and substituted aryl,

R₇ is chosen from:

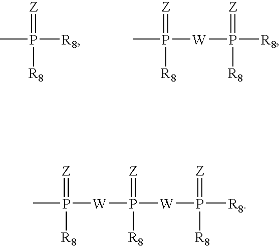

R₈ is selected from the group consisting of H, F, SR₉, and OR₉

R₉ is chosen from the group of H, alkyl, alkenyl, alkynyl, aryl, and hydroxyprotecting group,

Y is chosen from H, CH₃, and (CH₂)ₘR₅,

Z is chosen from O and S

W is chosen from CH₂, CF₂, CHF, and O,

m is 0, 1, 2, 3, or 4,

B is selected from adenine, guanine, cytosine, uracil, thymine, modified purines and pyrimidines such as inosin-9-yl, 2-amino-purin-9-yl, 2-amino-6-chloro-purin-9-yl, 2-6-diamino-purin-9-yl, 3-carboxamido-1,2,4-triazol-1-yl, 3-deaza-adenin-9-yl, 3-deaza-guanin-9-yl, 3-deaza-inosin-9-yl, 3-deaza-2-amino-purin-9-yl, 3-deaza-2-amino-6-chloro-purin-9-yl, 3-deaza-2,6-diamino-purin-9-yl, 7-deaza-adenin-9-yl, 7-deaza-guanin-9-yl, 7-deaza-inosin-9-yl, 7-deaza-2-amino-purin-9-yl, 7-deaza-2-amino-6-chloro-purin-9-yl, 7-deaza-2-6-diamino-purin-9-yl, 7-deaza-8-aza-adenin-9-yl, 7-deaza-8-aza-guanin-9-yl, 7-deaza-8-aza-inosin-9-yl, 7-deaza-8-aza-2-amino-purin-9-yl, 7-deaza-8-aza-2-amino-6-chloro-purin-9-yl, 7-deaza-8-aza-2-6-diamino-purin-9-yl, 8-aza-adenin-9-yl, 8-aza-guanin-9-yl, 8-aza-inosin-9-yl, 8-aza-2-amino-purin-9-yl, 8-aza-2-amino-6-chloro-purin-9-yl, 8-aza-2-6-diamino-purin-9-yl, 5-aza-thymin-1-yl, 5-aza-cytosin-1-yl, 5-aza-uracil-1-yl, 6-aza-thymin-1-yl, 6-aza-cytosin-1-yl, 6-aza-uracil-1-yl, 2-thiouracil-1-yl, 4-thiouracil-1-yl, 2-thiocytosine-1-yl, uracil-5-yl, 2-thiouracil-5-yl, 4-thiouracil-5-yl, substituted pyridine derivatives, 6-azauracil, azacytosine, five membered heterocycles with one to three heteroatoms (examples but not limited to triazole, thiazole, pyrazole, imidazole, pyrrole, thiophene, furane) substituted by at least one member selected from the group consisting of amines, substituted amines, amides, substituted amides, esters, halogens, alkyls, ethers; and pharmaceutically acceptable salts thereof and prodrugs thereof. In general, attachment may be at different positions in the ring at nitrogen or carbon. These ring systems may be substituted with halo, alkyl, substituted alkyl (F, Cl, Br, I, OH), NH₂, N₃, aryl, substituted aryl (F, Cl, Br, I, OH, NH₂), and aralkyl.

Definition of Terms

Listed below are definitions of various terms used to describe this invention. These definitions apply to the terms as they are used throughout this specification, unless otherwise limited in specific instances, either individually or as part of a larger group.

The term “alkyl” refers to straight or branched chain unsubstituted hydrocarbon groups of 1 to 20 carbon atoms, preferably 1 to 8 carbon atoms. The expression “lower alkyl” refers to unsubstituted alkyl groups of 1 to 4 carbon atoms. Alkyl groups may be substituted with halo (Cl, F, Br, I), OH, etc.

The term “aryl” refers to monocyclic or bicyclic aromatic hydrocarbon groups having 6 to 12 carbon atoms in the ring portion, such as phenyl, naphthyl, biphenyl and diphenyl and diphenyl groups, each of which may be substituted.

The term “acyl” refers to the residual moiety of a carboxylic acid group without the OH group of the acid and includes alkyl and acyl carboxylic acids. The alkyl group typically contains about 1-20 carbon atoms and more typically about 1-8 carbon atoms. The acyl group typically contains 6-12 carbon atoms. Examples of suitable acyl groups include acetyl and benzoyl.

Within the above-described definitions, certain embodiments are preferred. Preferred alkyl groups are lower alkyl groups containing 1 to about 8 carbon atoms, and more preferably 1 to about 5 carbon atoms, and can be straight, branched-chain or cyclic saturated aliphatic hydrocarbon groups.

Examples of suitable alkyl groups include methyl, ethyl and propyl. Examples of branched alkyl groups include isopropyl and t-butyl. An example of a suitable aralkyl group is phenethyl. Examples of suitable cycloalkyl groups typically contain 3-8 carbon atoms and include cyclopropyl, cyclobutyl, cyclopentyl and cyclohexyl. The aromatic or aryl groups are preferably phenyl and alkyl substituted aromatic groups (aralkyl) such as phenyl C₁₋₃ alkyl and benzyl.

“Prodrugs” are intended to include any covalently bonded carriers which release the active parent drug according to formula (I) in vivo when such prodrug is administered to a mammalian subject. Prodrugs of a compound of formula (I) are prepared by modifying functional groups present in the compound in such a way that the modifications are cleaved, either in routine manipulation or in vivo, to the parent compound.

Prodrug forms of the compounds bearing various nitrogen functions (amino, hydroxyamino, amide, etc.) may include the following types of derivatives where each R group individually may be hydrogen, substituted or unsubstituted alkyl, aryl, alkenyl, alkynyl, heterocycle, alkylaryl, aralkyl, aralkenyl, aralkynl, cycloalkyl or cycloalkenyl groups as defined earlier.

(a) Carboxamides, —NHC(O)R

(b) Carbamates, —NHC(O)OR

(c) (Acyloxy)alkyl Carbamates, NHC(O)OROC(O)R

(d) Enamines, —NHCR(═CHCO₂R) or —NHCR(═CHCONR₂)

(e) Schiff Bases, —N═CR₂

(f) Mannich Bases (from carboximide compounds), RCONHCH₂NR₂

Preparations of such prodrug derivatives are discussed in various literature sources (examples are: Alexander et al., J. Med. Chem. 1988, 31, 318; Aligas-Martin et al., PCT WO pp/41531, p.30). The nitrogen function converted in preparing these derivatives is one (or more) of the nitrogen atoms of a compound of the invention.

Prodrug forms of carboxyl-bearing compounds of the invention include esters (—CO₂R) where the R group corresponds to any alcohol whose release in the body through enzymatic or hydrolytic processes would be at pharmaceutically acceptable levels. Another prodrug derived from a carboxylic acid form of the invention may be a quaternary salt type

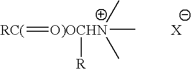

of structure described by Bodor et al., J. Med. Chem. 1980, 23, 469.

It is of course understood that the compounds of the present invention relate to all optical isomers and stereo-isomers at the various possible atoms of the molecule.

Pharmaceutically acceptable salts of the compounds of the present invention include those derived from pharmaceutically acceptable inorganic or organic acids. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include alkali such as sodium and ammonia.

The compounds of the present invention can be synthesized by persons skilled in the art once aware of the present disclosure without undue experimentation. Accordingly, detailed discussion of their preparation is not deemed necessary. Procedures are available in the chemical literature suitable for preparing the requisite sugars or nucleosides. Along these lines, see V. Samano and M. J. Robins. J. Org. Chem., 56, 7108 (1991); T. S. Lin, J. T. Zhu, G. E. Dulschman, Y.-C Cheng, and W. H. Prusoff. J. Med. Chem. 36, 353 (1993); D. Yu and M. d'Alareao. J. Med chem. 54, 3240 (1989); M. J. Bamford, P. L. Coe, and R. T. Walker. J. Med. Chem, 33, 2494 (1990); Y. S. Sanghri, B. Ross, R. Bharadwaj, and J.-J. Vasseur. Tetrahedron Lett. 35, 4697 (1994); H. M. Kissman, A. M. Hoffman, and M. J. Weiss. J. Med. Chem, 6, 407 (1963); L. Goldman, J. W. Marsico, J. J. Weiss, J. Med. Chem. 6, 410 (1963); N. N. Gerber. J. Med. Chem, 7, 204 (1964); M. C. Samano and M. J. Robins, Tetrahedron Lett. 32, 6293 (1991); W. S. Zielinski and L. E. Orgel. Nucleic Acids Res. 15, 1699 (1987); 2469 (1985); M. Moss. Ann., 666, 1982; H. Morisawa, T. Utagawa, S. Yamaneka and A. Yamazaki. Chem. Pharm. Bull., 29, 3191 (1981); I. Hechara, T. Murayama, H. Miki, and Y. Takatguka. Chem. Pharm. Bull. 25, 754 (1977); M. Unazawa and T. Eckstein. J. Org. Chem., 44, 2039 (1979); A. Zurk, S. VanCalenbergh, and P. Herdewijn. Tetrahedron Lett., 39, 5175 (1998); and M. J. Robins, S. D. Flaulrelak, A. E. Hernandez, S. Wnuk. Nucleosides Necleotides, 11, 821 (1992); disclosures of which are incorporated herein by reference.

Pursuant to the present invention, a study of the active site of HCV and other RNA polymerases as defined by x-ray crystallographic analysis indicates that many purine, pyrimidine and analogs thereof are tolerated in the part of the active site that binds the nucleic acid bases. It has also been determined according to the present invention that the part of the active site that binds the ribofuranose part of the nucleosides triphosphates can tolerate certain changes at the 2′ and 3′-hydroxyls of the ribofuranose ring. In addition, as found according to this invention, the hydroxyl groups, favorable interaction can occur with amino groups and with methylene amino groups. The amino groups can be substituted with alkyl and aralkyl groups. Therefore, the above disclosed compounds have been identified as inhibitors of RNA polymerase pursuant to this invention. Such inhibitors with sufficient potency will block the function of this enzyme preventing viral replication providing potential drugs for the treatment of diseases resulting from these viruses, such as hepatitis C, small pox, Ebola virus, West Nile virus, Polio, Coxsackie A and B, Rhino, and Echovirus.

Method of Hepatitis C RNA Dependent RNA Polymerase Assay

The polymerase activity assays were carried out according to the literature procedures with some modification. Briefly, the homopolymeric template including poly A/oligoT₁₆ bound to streptavidin-coated SPA beads (Amersham) was used to facilitate inhibitory compound screening. The reaction was incubated with various concentration of inhibitor, 0.5 μg of NS5B enzyme in a 50-μl reaction containing 0.1M Hepes (pH8.0), 1.75 mM MnCl₂, 4 mM dithiothreitol, 0.25 mg/ml rifampicin, 20 units of RNase inhibitor (Promega), and 10 μM UTP with 1 μCi ³H UTP (46.0 Ci/mmol, Amersham) for 2 hours at 30° C. After incubation, the reaction was terminated by addition of 100 μl of 0.12M EDTA (pH8.0) and diluted with 1 ml phosphate saline buffer (pH7.4). The incorporation of labeled UMP was determined by scintillation counting. The IC₅₀ of the inhibitor is defined as the concentration of the inhibitor at which 50% inhibition of the enzyme activity (control sample—no drug) was observed.

### Formulation

The compounds of the present invention can be administered by any conventional means available for use in conjunction with pharmaceuticals, either as individual therapeutic agents or in a combination of therapeutic agents. They can be administered alone, but generally administered with a pharmaceutical carrier selected on the basis of the chosen route of administration and standard pharmaceutical practice. The compounds can also be administered in conjunction with other therapeutic agents such as interferon (IFN), interferon α-2a, interferon α-2b, consensus interferon (CIFN), ribavirin, amantadine, remantadine, interleukine-12, ursodeoxycholic acid (UDCA), glycyrrhizin and silybum marianum.

The pharmaceutically acceptable carriers described herein, for example, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds of this invention can be administered by any conventional method available for use in conjunction with pharmaceuticals, either as individual therapeutic agents or in a combination of therapeutic agents.

The dosage administered well, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation of a drug powder mist. Other dosage forms are potentially possible such as administration transdermally, via patch mechanism or ointment.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants diluents, buffering agents, disintegrating agents, moistening agents, preservatives flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to an animal, particularly a human, in the context of the present invention should be sufficient to effect a therapeutic response in the animal over a reasonable time frame. One skilled in the art will recognize that dosage will depend upon a variety of factors including a condition of the animal, the body weight of the animal, as well as the severity and stage of the cancer.

A suitable dose is that which will result in a concentration of the active agent in a patient which is known to effect the desired response. The preferred dosage is the amount which results in maximum inhibition of cancer, without unmanageable side effects.

The size of the dose also will be determined by the route, timing and frequency of administration as well as the existence, nature, and extend of any adverse side effects that might accompany the administration of the compound and the desired physiological effect.

Useful pharmaceutical dosage forms for administration of the compounds according to the present invention can be illustrated as follows:

### Hard Shell Capsules

A large number of unit capsules are prepared by filling standard two-piece hard gelatine capsules each with 100 mg of powdered active ingredient, 150 mg of lactose, 50 mg of cellulose and 6 mg of magnesium stearate.

### Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

### Tablets

A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

### Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Moreover, the compounds of the present invention can be administered in the form of nose drops, or metered dose and a nasal or buccal inhaler. The drug is delivered from a nasal solution as a fine mist or from a powder as an aerosol.

The foregoing description of the invention illustrates and describes the present invention. Additionally, the disclosure shows and describes only the preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

What is claimed is:

1. A compound represented by the structure: 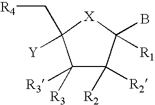
X is selected from the group consisting of O, S, and NR₆,
R₁ is selected from the group consisting of H, and (CH₂)ₘR₅
R₂, R_{2′}, R₃ and R_{3′} are independently selected from the group consisting of NO₂, N₃, and (CH₂)ₘR₅, OH
R₄ is selected from the group consisting of H, OR₆, SR₆, NR₆R₆ₐ, CN, C(O)OR₆, C(O)NR₆R₆ₐ, R₆, OR₇, and (CH₂)ₘR₇
R₅ is selected from the group consisting of H, halo, OR₆, SR₆, NR₆R₆ₐ, CN, C(O)OR₆, C(O)NR₆R₆ₐ, R₆, OR₇, and (CH₂)ₘR₇,
R₆ and R₆ₐ are individually selected from the group consisting of H, alkyl, substituted alkyl, alkenyl, substituted alkenyl, alkynyl, substituted alkynyl, aryl, and substituted aryl,
R₇ is selected from the group consisting of: 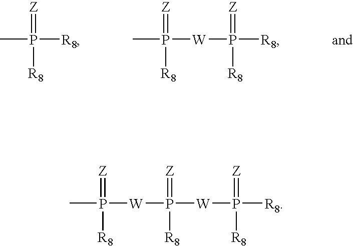
R₈ is selected from the group consisting of H, F, SR₉, and OR₉
R₉ is selected from the group consisting of H, alkyl, alkenyl, alkynyl, aryl, and hydroxyprotecting group,
Y is selected from the group consisting of H, CH₃, and (CH₂)ₘR₅,
Z is O or S
W is selected from the group consisting of CH₂, CF₂, CHF, and O,
m is 0, 1, 2, 3, or 4,
B is selected from the group consisting of adenine, guanine, cytosine, uracil, thymine, modified purines and pyrimidines, substituted pyridine derivatives, five membered heterocycles with one to three heteroatoms substituted by at least one member selected from the group consisting of amines, substituted amines, amides, substituted amides, esters, halogens, alkyls, and ethers; and being optionally substituted with at least one member selected from the group consisting of halo, alkyl, substituted alkyl, NH₂, N₃, aryl, and substituted aryl; and pharmaceutically acceptable salts thereof and prodrugs thereof.

2. A pharmaceutical composition containing at least one compound according to claim 1.

3. A method for inhibiting RNA polymerase in a patient by administering to the patient at least one compound according to claim 1 in an amount sufficient to inhibit viral RNA polymerase.

4. The method according to claim 3 wherein said RNA polymerase is selected from the group consisting of HCV, small pox, Ebola virus, and West Nile virus.

5. A method of treating a patient suffering from RNA viral infection by administering to the patient an effective amount of at least one compound according to claim 1.

6. The method according to claim 5 wherein the RNA viral infection is selected from the group consisting of HCV, HBV, and Rhino viral infection.

7. The compound of claim 1 wherein said modified purines and pyrimidines are selected from the group consisting of inosin-9-yl, 2-amino-purin-9-yl, 2-amino-6-chloro-purin-9-yl, 2-6-diamino-purin-9-yl, 3-carboxamido-1,2,4-triazol-1-yl, 3-deaza-adenin-9-yl, 3-deaza-guanin-9-yl, 3-deaza-inosin-9-yl, 3-deaza-2-amino-purin-9-yl, 3-deaza-2-amino-6-chloro-purin-9-yl, 3-deaza-2,6-diamino-purin-9-yl, 7-deaza-adenin-9-yl, 7-deaza-guanin-9-yl, 7-deaza-inosin-9-yl, 7-deaza-2-amino-purin-9-yl, 7-deaza-2-amino-6-chloro-purin-9-yl, 7-deaza-2-6-diamino-purin-9-yl, 7-deaza-8-aza-adenin-9-yl, 7-deaza-8-aza-guanin-9-yl, 7-deaza-8-aza-inosin-9-yl, 7-deaza-8-aza-2-amino-purin-9-yl, 7-deaza-8-aza-2-amino-6-chloro-purin-9-yl, 7-deaza-8-aza-2-6-diamino-purin-9-yl, 8-aza-adenin-9-yl, 8-aza-guanin-9-yl, 8-aza-inosin-9-yl, 8-aza-2-amino-purin-9-yl, 8-aza-2-amino-6-chloro-purin-9-yl, 8-aza-2-6-diamino-purin-9-yl, 5-aza-thymin-1-yl, 5-aza-cytosin-1-yl, 5-aza-uracil-1-yl, 6-aza-thymin-1-yl, 6-aza-cytosin-1-yl, 6-aza-uracil-1-yl, 2-thiouracil-1-yl, 4-thiouracil-1-yl, 2-thiocytosine-1-yl, uracil-5-yl, 2-thiouracil-5-yl, 4-thiouracil-5-yl, 6-azauracil, and azacytosine.

8. The compound of claim 1 wherein said five membered heterocycle is selected from the group consisting of triazole, thiazole, pyrazole, imidazole, pyrrole, thiophene, and furane.
